(19) United States
(12) Patent Application Publication
(10) Pub. No.: US 2024/0123188 A1
(43) Pub. Date: Apr. 18, 2024

(54) Medical Device Hub and System for Securing a First Medical Device Relative to a Second Medical Device

(71) Applicant: Becton, Dickinson and Company, Franklin Lakes, NJ (US)

(72) Inventor: Kevin M. Ryan, Whitehouse Station, NJ (US)

(21) Appl. No.: 17/968,244

(22) Filed: Oct. 18, 2022

(51) Int. Cl. A61M 25/00; A61M 25/06

(52) U.S. Cl. CPC A61M 25/0097; A61M 25/0606

(57) ABSTRACT

A medical device hub includes a monolithic housing defining a first fitting, a second fitting, and a mounting feature.

### BACKGROUND OF THE INVENTION

### Field of the Invention

The present disclosure relates generally to a medical device hub.

### Description of Related Art

Currently, about 20% of all patients with long-term in-dwelling intravenous (IV) catheters result in catheter dislodgement injuries to patients. Some injuries result in the IV catheter inadvertently penetrating the wrong part of the patient's body. Other injuries arise from the IV catheter base becoming sutured (known as primary retention) to the patient's skin adjacent to the insertion site. One root-cause of these issues stems from inadvertent yanking or pulling of the IV catheter infusion extension line, which induces a tension on the of the indwelling IV catheter line, which in turn leads to IV catheter dislodgement from the patient's IV insertion site. This inadvertent yanking or pulling of the IV catheter extension line typically occurs when the patient mistakenly moves away from the IV Infusion source, such as walking away from an IV bag hanging on an IV pole or an IV pole mounted pump. Alternatively, pulling of the IV catheter extension line can also occur if the extension line becomes caught on the patient's bed or other objects in the vicinity.

These dislodgement injuries are problematic due to the increased risk of infection and surgical procedures necessary to treat infection and or replace the IV catheter.

Current solutions to the above-mentioned problems of IV catheter dislodgement have yielded marginal success. One approach includes using copious amounts of tape around the IV catheter line and or IV extension line in attempts to hold the line (make-shift secondary retention) to the patient's body, such that inadvertent yanking or pulling forces/loads do not translate down directly to the IV insertion site or the primary retention suture area. This approach has a low demonstrated effectiveness, which is evident in high percentage of the current IV catheter dislodgement related injuries. Further, using copious amounts of tape by the clinician is not ideal due to the time and inconstant way the tape is applied.

Other solutions such as secondary retention wearable strap devices have been tried. However, these devices also tend to have low effectiveness and adoption due to the IV catheter not having an adequate attachment or interface feature/s between the catheter and the secondary retention device.

Accordingly, those skilled in the art continue with research and development efforts in the field of medical device hubs and systems for securing a first medical device relative to a second medical device.

### SUMMARY OF THE INVENTION

Disclosed is a medical device hub.

In one example, the medical device hub includes a monolithic housing defining a first fitting, a second fitting, and a mounting feature.

The first fitting may be configured to removably couple with a first medical device. The first fitting may include a threaded portion configured to threadedly engage with a first medical device. The first fitting may be an ISO-80369-7 Male Fluid Fitting.

The second fitting may be configured to removably couple with a second medical device. The second fitting may include a threaded portion configured to threadedly engage with a second medical device. The second fitting may be an ISO-80369-7 Female Fluid Fitting.

The mounting feature may be a flanged structure. The mounting feature may define at least one engagement feature. The housing may be of a polymeric material.

The medical device hub may include a second mounting feature defined by the housing. The second mounting feature may define at least one engagement feature. The medical device hub may include a fluid channel defined by the housing. The fluid channel may be in fluid communication with a first medical device and a second medical device.

Also disclosed is a system for securing a first medical device relative to a second medical device.

In one example, the system includes a first medical device, a second medical device in fluid communication with the first medical device, and a medical device hub positioned between the first medical device and the second medical device. The medical device hub includes a monolithic housing defining a first fitting, a second fitting in fluid communication with the first fitting, and a mounting feature.

The medical device hub of the system may further include a fluid channel defined by the housing. The first medical device may be in fluid communication with the second medical device by way of the fluid channel. The first medical device may be an indwelling IV Catheter Hub and the second medical device may be a needleless connector. The system may further include an IV catheter lumen coupled to the first medical device and an IV extension line coupled to the second medical device.

The medical device hub of the system may include a mounting bracket removably coupled to the mounting feature of the medical device hub. The mounting bracket may include a bracket engagement structure configured to nest with the mounting feature of the medical device hub. The mounting feature may define at least one engagement feature. The mounting bracket engagement structure may include at least one mounting bracket engagement feature. The engagement feature may be configured to nest with the mounting bracket engagement feature.

The system may further include a retention device removably coupled to the mounting bracket. The retention device may be removably affixed to a user. The retention device may be one or more of an arm band and a strap. The mounting bracket may include at least one retention device engagement feature and wherein the retention device may be removably engageable with the retention device engagement feature.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a medical device hub;

FIG. 2 is a perspective cross-sectional view of the medical device hub of FIG. 1;

FIG. 3 is a perspective schematic of a system for securing a first medical device relative to a second medical device;

FIG. 4 is a perspective schematic of a portion of the system of FIG. 3;

FIG. 5 is a perspective cross-sectional schematic of a portion of the system of FIG. 3; and

FIG. 6 is a perspective schematic of a portion of the system of FIG. 5.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

Reference herein to “one or more examples” means that one or more feature, structure, or characteristic described in connection with the example is included in at least one implementation. The phrase “one or more examples” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

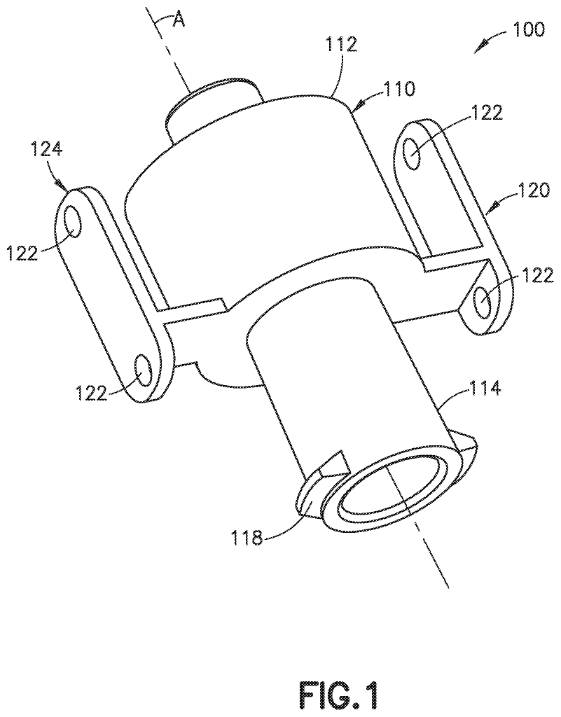

Referring to FIG. 1, disclosed is a medical device hub 100. The medical device hub 100 is a single, monolithic piece configured to couple with two or more medical devices. In one example, the medical device hub 100 is configured to removably couple a first medical device 220 with a second medical device 230 such that the first medical device 220 is in fluid communication with the second medical device 230. The medical device hub 100 is designed for use on IV luer catheter hubs. The disclosed design incorporates an IV luer male luer, a Female luer, and one or more mounting bracket features in a single molded part. This combination of features allow the medical device hub 100 to connect to IV catheter accessories such as primary and or secondary catheter pull load retention devices.

In one aspect, the medical device hub 100 is designed to absorb at least a portion of any tension or other force/load applied to the first medical device 220 and/or second medical device 230 in the event that a portion of either medical device 220, 230 or another medical device coupled with one or more of the first medical device 220 and/or second medical device 230 is inadvertently pulled or becomes entangled with an external structure. By absorbing at least a portion of any tension or other force/load, first medical device 220, second medical device 230, and/or any other medical device coupled with one or more of the first medical device 220 and/or second medical device 230 is less susceptible to dislodgement and/or damage.

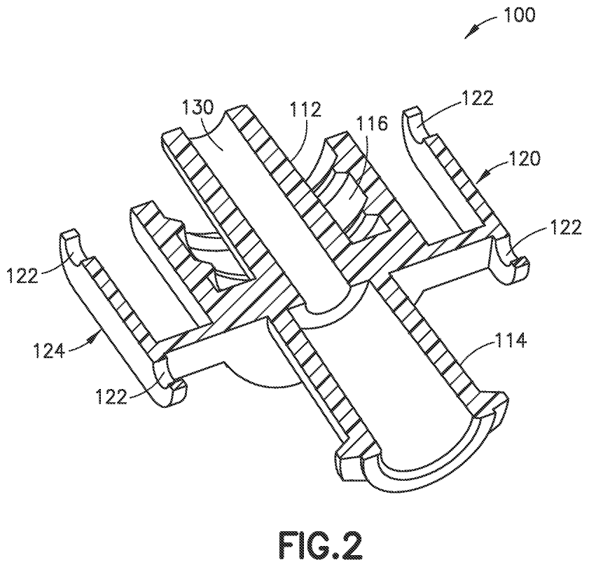

Still referring to FIG. 1 and FIG. 2, in one or more examples, medical device hub 100 includes a monolithic housing 110. The monolithic housing 110 may be a single molded piece having various engagement and coupling features. The monolithic housing 110 may be comprised of any material having requisite material properties for absorbing tension shearing, or other pulling forces/loads while maintain engagement with two or more medical devices. In one example, the housing 110 is comprised of a polymeric material.

In one example, the monolithic housing 110 defines a first fitting 112. The first fitting 112 is configured to removably couple with a first medical device 220. The first fitting 112 may be sized and shaped to snugly fit and nest with the first medical device 220. In one example, the first fitting 112 includes a threaded portion 116 configured to threadedly engage with a first medical device 220. The threaded portion 116 may define a threaded channel surrounding a fluid channel 130 (described below) for receiving the first medical device 220. For example, the threaded portion 112 of the first fitting 112 is located on an inside surface of the first fitting 112 so that when it is threadedly engaged with the first medical device 220, the first medical device 220 is simultaneously partially received within a portion of the medical device hub 100 while surrounding another portion of the medical device hub 100. In one specific, non-limiting example, the first fitting 112 is an ISO-80369-7 Male Fluid Fitting.

Still referring to FIG. 1 and FIG. 2, the monolithic housing 110 defines a second fitting 114. The second fitting 114 is in fluid communication with the first fitting 112. The second fitting 114 is configured to removably couple with a second medical device 230. The second fitting 114 may be sized and shaped to snugly fit and nest with the second medical device 230. In one example, the second fitting 114 comprises a threaded portion 118 configured to threadedly engage with a second medical device 230. For example, the threaded portion 118 of the second fitting 114 is located on an outside surface of the second fitting 114 so that when it is threadedly engaged with the second medical device 230, the threaded portion 118 is housed within the second medical device 230. Further, while in threaded engagement, the second medical device 230 is simultaneously partially received within a portion of the medical device hub 100 while surrounding another portion of the medical device hub 100. In one specific, non-limiting example, the second fitting 114 is an ISO-80369-7 Female Fluid Fitting.

Still referring to FIG. 1 and FIG. 2, the monolithic housing 110 defines a mounting feature 120. In one example, the mounting feature 120 is a flanged structure. The mounting feature 120 may define at least one engagement feature 122. As illustrated in FIGS. 1 and 2, the mounting feature 120 may define more than one engagement feature 112. The mounting feature 120 is configured to removably couple with a secondary structure, such as a retention device 250 or a mounting bracket 240, to further secure the medical device hub 100 and associated medical devices 220, 230 to a patient or user 210.

The medical device hub 100 may further include a second mounting feature 124 defined by the housing 110. The second mounting feature 124 may be laterally opposed from the mounting feature 120 relative to a central axis A. The second mounting feature 124 may mirror the mounting feature 120 such that they are substantially equally sized and shaped and may define at least one engagement feature 122. For example, as shown in FIG. 1, each of the mounting feature 120 and the second mounting feature 124 is a generally elongated structure parallel to the central axis A and includes an engagement feature 122 on either end of the elongated structure. However, it is contemplated that the mounting feature 120 and the second mounting feature 124 may have different shapes and different quantities of engagement feature 122 without impacting the functionality of the medical device hub 100.

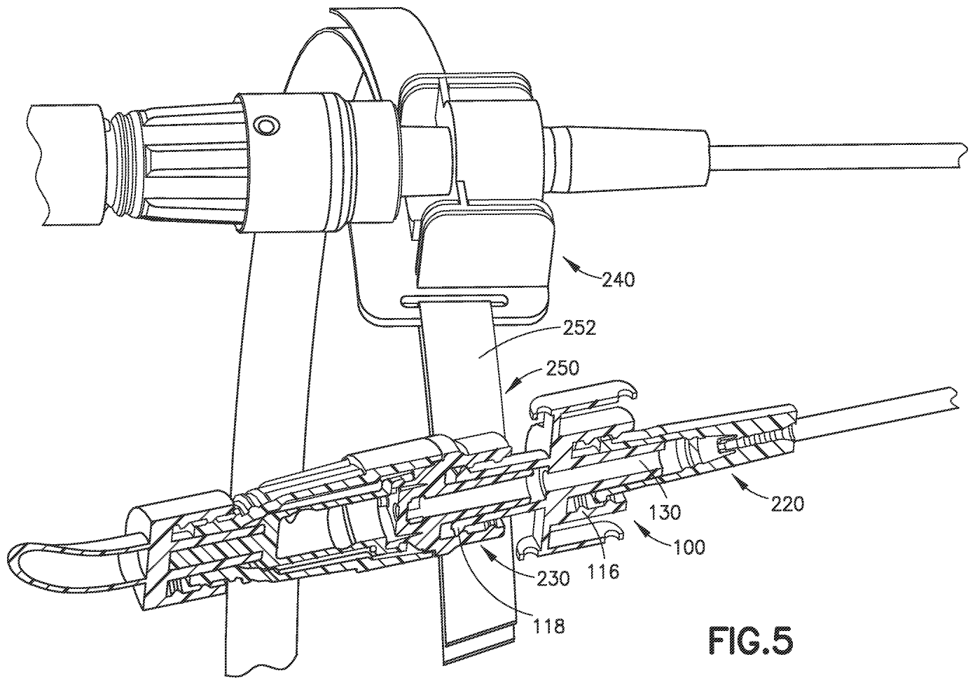

Referring to FIG. 2, in one or more example, the medical device hub 100 further includes a fluid channel 130 defined by the housing 110. The fluid channel 130 is in fluid communication with a first medical device 220 and a second medical device 230. The fluid channel 130 may be configured to nest within the first medical device 220, see FIG. 5, such that it is in fluid communication with the first medical device without impacting the flow of fluid between the first medical device 220 and the second medical device 230. Similarly, as also illustrated in FIG. 5, the fluid channel 130 is configured to nest with the second medical device 230 such that they are in fluid communication without impacting the flow of fluid between the first medical device 220 and the second medical device 230. In one example, the fluid channel 130 is substantially defined by the first fitting 112 and has a diameter that aligns with an inside diameter of a portion of the second medical device 230 that nests within the second fitting 114.

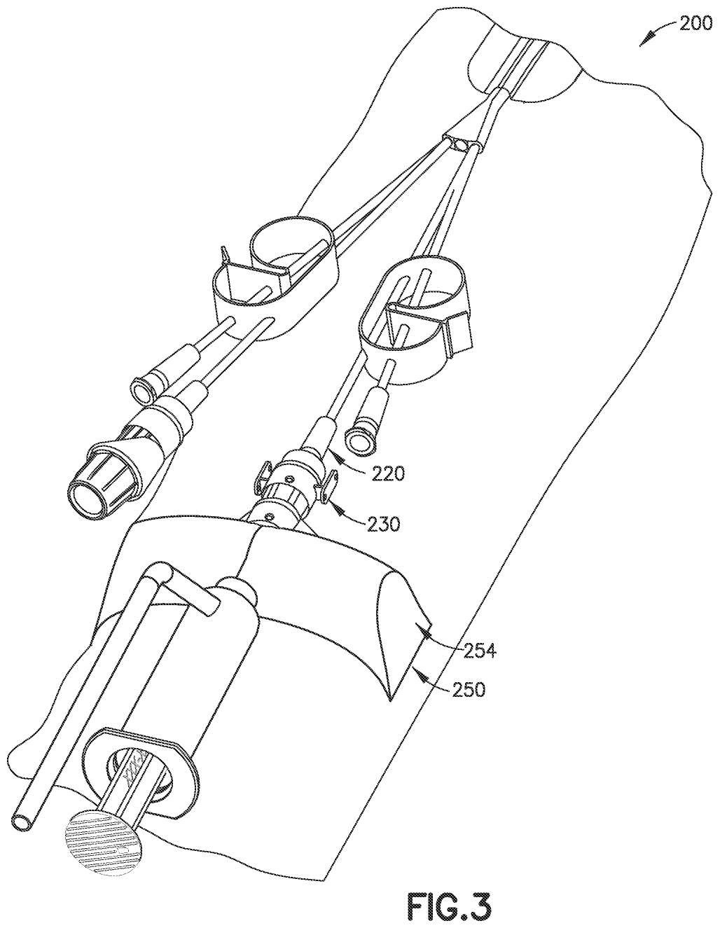

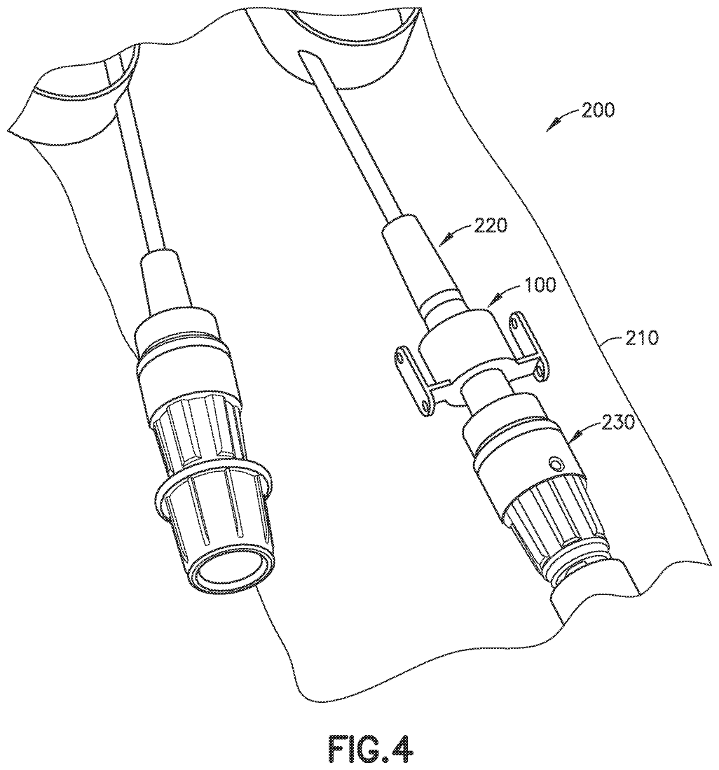

Referring to FIGS. 3, 4, and 5, disclosed is a system 200 for securing a first medical device 220 relative to a second medical device 230. The system 200 is designed to securely place a first medical device 220 in fluid communication with a second medical device 230 in such a manner to maintain structural integrity in the event a tension force or load due to pulling or tugging is applied to one or more of the first medical device 220 and the second medical device 230. Therefore, the two medical devices 220, 230 are less susceptible to becoming uncouples and/or out of fluid communication than if they were directly coupled together.

As illustrated in FIGS. 3, 4, and 5, the system 200 includes a first medical device 220 and a second medical device 230 in fluid communication with the first medical device 220. The first medical device 220 and the second medical device 230 may be components of a catheter, such as an intravenous (IV) catheter. In one example, the first medical device 220 is an indwelling IV catheter hub. In another example, the second medical device 230 is a needleless connector. The system 200 may further include other catheter components, including but not limited to an IV catheter lumen, an IV extension line coupled to the needleless connector.

Still referring to FIGS. 3, 4, and 5, the system 200 includes a medical device hub 100 positioned between and in fluid communication with the first medical device 220 and the second medical device 230. As shown and described herein, the medical device 100 includes a monolithic housing 110 defining a first fitting 112, a second fitting 114 in fluid communication with the first fitting 112, and a mounting feature 120. The mounting feature 120 may define at least one engagement feature 122 or may define two or more of engagement feature 122.

In one or more examples, the medical device hub 100 further includes a fluid channel 130 defined by the housing 110. In one example, the first medical device 220 is in fluid communication with the second medical device 230 by way of the fluid channel 130.

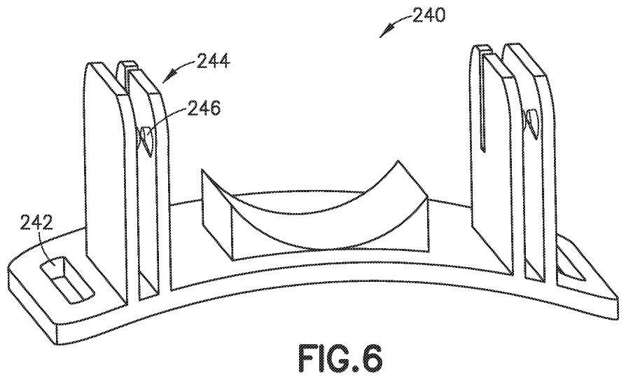

Referring to FIGS. 5 and 6, in one or more examples, the system 200 may further include a mounting bracket 240 removably coupled to the mounting feature 120 of the medical device hub 100. The mounting bracket 240 may include a bracket engagement structure 244 configured to nest with the mounting feature 120 of the medical device hub 100. The mounting bracket engagement structure 244 may include at least one mounting bracket engagement feature 246, or a plurality of engagement feature 246. The engagement feature 122 of the mounting feature 120 may be configured to nest with the mounting bracket engagement feature 246. In one example, the mounting bracket 240 is a single, monolithic piece having the above-mentioned features.

The system 200 may further include a retention device 250. In one example, the retention device 250 is removably coupled to the mounting bracket 240, see FIG. 5. The retention device 250 may be removably affixed to a user 210. The retention device 250 may be any structure configured to comfortably and safely secure one or more medical device and the medical device hub 100 to a user 210. For example, the retention device 250 may be an arm band 252 or a strap 254. The retention device 250 may be secured to itself using a hook and loop configuration, buckle, magnet, clip, or other means for adjustably and removably securing to a user 210. In one specific, non-limiting example, the medical device hub 100 can be attached in-between an indwelling IV catheter hub that exits an IV Catheter Lumen and an IV Needle Free Connector an IV Extension Line. In this configuration, the medical device hub 100 can be attached to a retention device 250 and/or taped directly to the user 210 such that it can more effectively transfer the full yanking or pulling load forces from the IV Extension Line to the patient's body that is further away from the IV Catheter Insertion Site.

The mounting bracket 240 may include at least one retention device engagement feature 242 for removably coupling with the retention device 250. The mounting bracket 240 may further include two or more of retention device engagement feature 242. In one example, the retention device 250 is removably engageable with the retention device engagement feature 242.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

The invention claimed is:

1. A medical device hub comprising:
a monolithic housing defining:
a first fitting;
a second fitting in fluid communication with the first fitting; and
a mounting feature.

2. The medical device hub of claim 1, wherein the first fitting is configured to removably couple with a first medical device.

3. The medical device hub of claim 1, wherein the first fitting comprises a threaded portion configured to threadedly engage with a first medical device.

4. The medical device hub of claim 1, wherein the first fitting is a male luer fitting.

5. The medical device hub of claim 1, wherein the second fitting is configured to removably couple with a second medical device.

6. The medical device hub of claim 1, wherein the second fitting comprises a threaded portion configured to threadedly engage with a second medical device.

7. The medical device hub of claim 1, wherein the second fitting is a female luer fitting.

8. The medical device hub of claim 1, wherein the mounting feature is a flanged structure.

9. The medical device hub of claim 1, wherein the mounting feature defines at least one engagement feature.

10. The medical device hub of claim 1, wherein the housing is comprised of a polymeric material.

11. The medical device hub of claim 1, further comprising a second mounting feature defined by the housing.

12. The medical device hub of claim 11, wherein the second mounting feature defines at least one engagement feature.

13. The medical device hub of claim 1, further comprising a fluid channel defined by the housing.

14. The medical device hub of claim 13, wherein, when the medical device hug is coupled with a first medical device and a second medical device, the fluid channel is in fluid communication with the first medical device and the second medical device.

15. A system for securing a first medical device relative to a second medical device, the system comprising:
a first medical device;
a second medical device in fluid communication with the first medical device; and
a medical device hub positioned between the first medical device and the second medical device, the medical device hub comprising:
a monolithic housing defining:
a first fitting;
a second fitting in fluid communication with the first fitting; and
a mounting feature.

16. The system of claim 15, wherein the medical device hub further comprises a fluid channel defined by the housing.

17. The system of claim 16, wherein the first medical device is in fluid communication with the second medical device by way of the fluid channel.

18. The system of claim 15, wherein the first medical device is an indwelling IV catheter hub.

19. The system of claim 15, further comprising an IV catheter lumen coupled to the first medical device.

20. The system of claim 15, wherein the second medical device is a needleless connector.

21. The system of claim 15, further comprising an IV extension line coupled to the second medical device.

22. The system of claim 15, further comprising a mounting bracket removably coupled to the mounting feature of the medical device hub.

23. The system of claim 22, wherein the mounting bracket comprises a bracket engagement structure configured to nest with the mounting feature of the medical device hub.

24. The system of claim 23, wherein:
the mounting feature defines at least one engagement feature;
the mounting bracket engagement structure comprises at least one mounting bracket engagement feature; and
wherein the engagement feature is configured to nest with the mounting bracket engagement feature.

25. The system of claim 22, further comprising a retention device removably coupled to the mounting bracket.

26. The system of claim 25, wherein the retention device is removably affixed to a user.

27. The system of claim 25, wherein the retention device is one or more of an arm band and a strap.

28. The system of claim 25, wherein the mounting bracket comprises at least one retention device engagement feature and wherein the retention device is removably engageable with the retention device engagement feature.
